(19) United States
(12) Patent Application Publication
(10) Pub. No.: US 2008/0125731 A1
(43) Pub. Date: May 29, 2008

(54) DEVICE FOR PROPHYLACTICS AND TREATMENT OF BALANITIS

(76) Inventor: Dmitri Dozortsev, Katy, TX (US)

Correspondence Address: DMITRI DOZORTSEV, 858 SHILLINGTON DR, KATY, TX, 77450, US

(21) Appl. No.: 11/934,708

(22) Filed: Nov. 2, 2007

Related U.S. Application Data
(60) Provisional application No. 60/856,153, filed on Nov. 2, 2006.

(51) Int. Cl. A61F 5/44

(52) U.S. Cl. 604/349

(57) ABSTRACT

The invention provides a disposable ring made of a moisture absorbing material that is placed on a glance of the penis in a manner that creates an aerated space between the glans and the foreskin and prevents the direct contact between glans and foreskin or underwear.

### CROSS-REFERENCE TO RELATED APPLICATIONS

Claims the benefit of earlier filed provisional application 60/856,153 filed on Nov. 2, 2006.

### TECHNICAL FIELD OF INVENTION

The present invention relates to an inflammation treatment apparatus. More particularly, the present invention relates to an inflammation treatment apparatus for treatment of balanitis and balanoposthitis.

### BACKGROUND OF THE INVENTION

Balanitis and balanoposthitis are common inflammatory disorders affecting primarily uncircumcised men. Although there is often an underlying specific infectious agent, such as virus or bacteria, there are strong evidences pointing to anaerobic conditions in the space between foreskin and glans of the penis as the main causative factor maintaining the inflammation.

Current treatments include a variety of ointments and creams, usually with some antibacterial or anti-viral properties.

### However, in many cases they are ineffective or inflammation comes back once treatment is stopped.

The proposed invention represents a less expensive option that in many cases is more effective than medication or may be used as a supplement to the medication.

### SUMMARY OF THE INVENTION

The invention represents a disposable ring made of the moisture absorbing material that is placed on a glance of the penis in a manner that creates an aerated space between the glans and the foreskin and prevents the direct contact between glans and foreskin.

In circumcised men, the device can be used to prevent the frictions between exposed skin of the penis and underwear.

This device is preferentially disposable and is replaced as it becomes saturated with moisture. In patient testing the device cured a chronic torpid inflammation, that was not sensitive to steroid hormones or antibiotics, within several days.

### BRIEF DESCRIPTION OF THE DRAWING

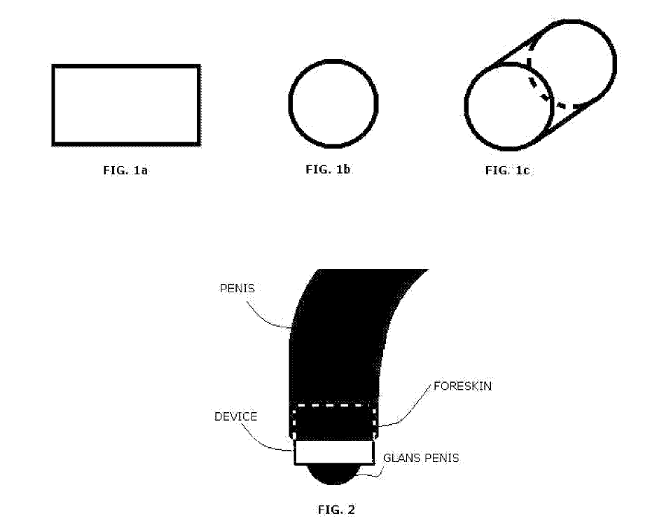

In the drawing, FIG. 1a represent the side view, FIG. 1b represents the front view, and FIG. 1c a general view of the device.

The FIG. 2 shows the preferred application of the device where it is inserted under the foreskin while protruding outside, thereby creating aerated envelope between glans and the foreskin.

### DETAILED DESCRIPTION OF THE INVENTION

Referring to the drawing that is self-explanatory, it can be understood that the present invention represents a bandage that absorbs moisture, aerates the space between the foreskin and the glance penis and prevents the direct contact between the glans and foreskin.

The bandage's shape can be further modified to make it better fit the shape of the glans.

### References

U.S. Pat. No. 6,967,261
Masfari A N, Kinghorn G R, Duerden B I. Anaerobes in genitourinary infections in men.
Br J Vener Dis. 1983 Aug;59(4):255-9.

What is claimed is:

1. A device made of moisture absorbing material that wraps around the glans of penis and protrudes outside creating an aerated envelope and preventing the contact between the foreskin and glans of penis or glans of penis and underwear.

2. The device of claim 1 wherein such device is disposable

3. The device of claim 1 wherein such device is treated with chemically active substance.

4. The device of claim 3 wherein active chemical substance has antimicrobial or antivirus properties.

5. The device of claim 1 wherein it is shaped to accommodate frenulum.

6. The device of claim 1 wherein it is held in place by foreskin.

7. The device of claim 1 wherein it is made of stretching material

8. The device of claim 1 wherein it is shaped to be held in place by bending its distal edge over the glans penis into the sulcus.

9. The device of claim 1 wherein it is manufactured to fit different sizes.

10. The device of claim 1 wherein it is manufactured as a flat piece of tissue, that is wrapped around the glans with the size adjusted by user using some type of the sticky material.

11. The device of claim 1 wherein it is shaped to follow the shape of the glance.

12. The device of claim 7 wherein it is held in place by elastic properties of the material

13. The device of claim wherein it shaped and distributed as tube

14. The device of claim 13 wherein it is distributed as a continued tube precut at the intervals.
